SEQUENCE LISTING (8 sequences)

SEQ ID NO: 1 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 137 aa, synthetic construct
GIPPHVQASVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - protein, 610 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQASVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 21 aa, synthetic construct
AGGGGSGGGGSGGGGSGGGGS

SEQ ID NO: 6 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 137 aa, synthetic construct
GIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 611 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

(19) United States
(12) Patent Application Publication
(10) Pub. No.: US 2026/0242454 A1
(43) Pub. Date: Aug. 20, 2026

(54) Bifunctional fusion protein, and preparation method and use thereof

(71) Applicants: Fusion Bioscience Inc., Shanghai (CN); Huapu Shijiazhuang Pharmaceutical Co., Ltd., Shijiazhuang (CN)

(72) Inventors: Xinzhong WANG, Shanghai (CN); Bixiong XU, Shanghai (CN); Libo ZHOU, Shanghai (CN)

(73) Assignees: Fusion Bioscience Inc., Shanghai (CN); Huapu Shijiazhuang Pharmaceutical Co., Ltd., Shanghai (CN)

(21) Appl. No.: 18/832,538

(22) PCT Filed: Jan. 22, 2023
(86) PCT No.: PCT/CN2023/073446

§ 371 Date: Jul. 24, 2024

(30) Foreign Application Priority Data
Jan. 25, 2022 (CN) 202210089078.X
Jan. 6, 2023 (CN) 202310020371.5

(51) Int. Cl. C07K 16/22; A61K 31/138; A61K 31/175; A61K 31/282; A61K 31/475; A61K 31/519; A61K 31/573; A61K 31/675; A61K 31/704; A61K 33/00; A61K 33/243; A61K 38/14; A61P 35/00

(52) U.S. Cl. CPC C07K 16/22; A61K 31/138; A61K 31/175; A61K 31/282; A61K 31/475; A61K 31/519; A61K 31/573; A61K 31/675; A61K 31/704; A61K 33/00; A61K 33/243; A61K 38/14; A61P 35/00; C07K 2317/31

(57) ABSTRACT

The bifunctional fusion protein comprises an anti-VEGF fragment and an anti-TGF-β fragment, the anti-VEGF fragment comprises SEQ ID Nos. 1 and 2 or a polypeptide fragment having more than 90% sequence identity thereto, and the anti-TGF-β fragment comprises SEQ ID No. 3 or a polypeptide fragment having more than 90% sequence identity thereto. The present invention also provides a method for preparing the bifunctional fusion protein, and a use of the bifunctional fusion protein in the preparation of drugs for the treatment of diseases associated with VEGF and/or TGF-β expression. The bifunctional fusion protein provided by the present invention is not prone to fragmentation during the purification process and exhibits good stability. Therefore, the purification efficiency can be improved, the production cost can be reduced, making it highly promising for broad applications and commercial value.

### FIELD OF THE INVENTION

The present disclosure relates to the technical field of biotechnology, in particular to a bifunctional fusion protein, as well as a preparation method and a use thereof.

### BACKGROUND OF THE INVENTION

In the tumor microenvironment, transforming growth factor beta (TGF-β) plays an important role in immunosuppression. TGF-β regulates the production and function of many types of immune cells. It controls adaptive immunity by directly promoting the proliferation of Treg cells and suppressing the production and function of effector T cells (Teff) and antigen-presenting dendritic cells (DC). Additionally, it controls the innate immune system by inhibiting natural killer cells (NK cells) and regulating the complex behavior of macrophages and neutrophils, forming a negative immune regulation network. It has been shown that angiogenesis is one of the key factors in the growth of malignant tumors, and inhibition of vascular endothelial growth factor (VEGF), a highly specific pro-angiogenic factor that promotes the growth of endothelial cells, can effectively suppress tumor growth. Anti-angiogenic drugs, represented by VEGF antibodies, have become a focus in anti-tumor drug research and development due to their good specificity and efficacy, as well as low likelihood of resistance.

Bevacizumab (Avastin), developed by Genentech, a subsidiary of Roche, is a recombinant humanized immunoglobulin G1 (IgG1) monoclonal antibody. It can bind to VEGF-A and inhibit its interaction with VEGF receptor-2 (VEGFR-2), thereby suppressing the biological effects of VEGF, including influence on vascular permeability and proliferation, and endothelial cell migration and survival. This leads to the inhibition of tumor angiogenesis, growth, and metastasis. A variety of small and large molecule VEGF antagonists in combination with PD-1 or PD-L1 inhibitors have demonstrated remarkable efficacy in several cancers, including advanced liver cancer, and have become one of the important combination therapies for cancer treatment so far.

The negative immunosuppressive tumor microenvironment is a widely recognized major factor hindering and limiting the clinical efficacy of immune checkpoint drugs, including inhibitors of programmed cell death receptor 1 (PD-1) and programmed death ligand 1 (PD-L1). The negative impact of the tumor microenvironment is particularly pronounced in solid tumors, making it a key focus in new drug development with respect to response rate and resistance. For example, M7824, developed by Merck Serono, a subsidiary of Merck, is a bifunctional fusion protein consisting of an anti-PD-L1 monoclonal antibody fused to the extracellular domain of the human TGF-β receptor II (TGF-β RII). It can antagonize both PD-L1 and TGF-β. M7824 showed good activity in early clinical studies but did not demonstrate advantages in the phase III clinical trial. It is evident that inhibiting TGF-β alone is not sufficient to overcome the negative regulation of the tumor microenvironment.

In addition, the existing bifunctional fusion proteins containing TGF-β receptor, taking Merck's M7824 as an example, are prone to fragmentation during the purification process because of the inherent physiological properties of TGF-β receptor, resulting in greatly reduced purification efficiency and increased production cost, and ultimately affecting the efficacy of the drug. Moreover, the clinical dosage of the anti-PD-L1 antibody is much larger than that of the anti-PD-1 antibody, which further increases the clinical development difficulty of M7824 and limits its future clinical application.

### SUMMARY OF THE INVENTION

The present invention provides a bifunctional fusion protein, a preparation method and a use thereof, which offers a new technical approach to efficiently target the tumor microenvironment and enhances the therapeutic efficacy of the combination therapy with PD-1 or PD-L1 inhibitors.

One aspect of the present invention is to provide a bifunctional fusion protein, the bifunctional fusion protein comprises an anti-VEGF fragment and an anti-TGF-β fragment.

The anti-VEGF fragment comprises a heavy chain, wherein the heavy chain comprises an amino acid mutation at least at position 453 compared to an original heavy chain; and/or, the anti-TGF-β fragment comprises an amino acid mutation at least at position 8 compared to an original anti-TGF-β fragment.

Another aspect of the present invention is to provide an isolated polynucleotide encoding the bifunctional fusion protein as described above.

Another aspect of the present invention is to provide a construct, wherein the construct comprises the isolated polynucleotide as described above.

Another aspect of the present invention is to provide an expression system, wherein the expression system comprises the construct as described above or a genome incorporating an exogenous polynucleotide as described above.

Another aspect of the present invention is to provide a method for preparing the bifunctional fusion protein as described above, wherein the method comprises: culturing the expression system as described above under suitable conditions to express the bifunctional fusion protein, and performing isolation and purification to provide the bifunctional fusion protein.

Another aspect of the present invention is to provide a use of the bifunctional fusion protein as described above in the preparation of drugs. The drugs are used for the treatment of diseases associated with VEGF and/or TGF-β expression, preferably for the treatment of tumors. The treatment of tumors includes inhibiting tumor angiogenesis, growth, and metastasis, suppressing the generation, proliferation, migration, and invasion of tumor cells, and promoting tumor cell apoptosis.

Another aspect of the present invention is to provide a pharmaceutical composition, wherein the pharmaceutical composition comprises the bifunctional fusion protein as described above, or the expression system as described above, or a culture of the expression system as described above.

The bifunctional fusion protein and the biological materials related to the bifunctional fusion protein can be synthesized artificially or by first synthesizing their encoding gene followed by biological expression. In a specific embodiment, the bifunctional fusion protein and the biological materials related to the bifunctional fusion protein in the present invention are produced by introducing the gene into E. coli or yeast, mammalian cells such as CHO cells, HEK293 cells, etc., and utilizing the expression of the E. coli, yeast, or mammalian cells, etc.

The present invention has the following advantages:
(1) by deleting the K amino acid residue at position 453 of the heavy chain of the anti-VEGF fragment, and by mutating the K amino acid residue at position 8 to A (i.e., K→A mutation) of the anti-TGF-β fragment, the obtained bifunctional fusion protein in the present invention features good stability and is not prone to fragmentation during the purification process. Therefore, the purification efficiency is improved and the production cost is reduced. The molecular weight of the purified bifunctional fusion protein in the present invention is about 179 kD, and the purity is greater than 98%.
(2) The bifunctional fusion protein in the present invention has a very good affinity, and is capable of binding to recombinant human TGF-β 1/2/3, with an affinity basically comparable to that of M7824. The bifunctional fusion protein is also capable of binding to recombinant human VEGF, with an affinity basically comparable to that of Avastin. Furthermore, the bifunctional fusion protein of the present invention is capable of binding to both recombinant human TGF-β 1/2/3 and VEGF, therefore, it can be applied to the treatment of diseases associated with VEGF and/or TGF-β expression.
(3) The in vivo anti-tumor efficacy experiment shows that the bifunctional fusion protein described herein can effectively inhibit the growth of tumors in mice and can be used in the treatment of tumors.

### BRIEF DESCRIPTION OF DRAWINGS

FIG. 1 shows results of the reducing electrophoresis identification experiment of the bifunctional fusion protein according to Example 4 of the present invention.

FIG. 2 shows results of the non-reducing electrophoresis identification experiment of the bifunctional fusion protein according to Example 4 of the present invention.

FIG. 3 shows stability results of the bifunctional fusion protein by using the reducing electrophoresis identification experiment according to Example 4 of the present invention.

FIG. 4 shows results of the ELISA assay of TGF-β1 according to Example 5 of the present invention.

FIG. 5 shows results of the ELISA assay of TGF-β2 according to Example 5 of the present invention.

FIG. 6 shows results of the ELISA assay of TGF-β3 according to Example 5 of the present invention.

FIG. 7 shows results of the ELISA assay of VEGF according to Example 6 of the present invention.

FIG. 8 shows results of the ELISA assay for simultaneous detection of TGF-β and VEGF binding according to embodiment 7 of the present invention.

FIG. 9 shows results of the experiment inhibiting VEGF-induced HUVEC proliferation according to Example 8 of the present invention.

FIG. 10 shows results of the experiment inhibiting TGF-β-induced human breast cancer MDA-MB-231 cell migration according to Example 9 of the present invention.

FIG. 11 shows statistical results of the experiment inhibiting TGF-β-induced human breast cancer MDA-MB-231 cell migration according to Example 9 of the present invention.

FIG. 12 shows experimental results of the bifunctional fusion protein's neutralizing effect on TGF-β according to Example 10 of the present invention, wherein Control represents PBS, Control 2 represents PBS+TGF-β1, Control 3 represents 10 nM HSP088-01+TGF-β1, Test 1 represents 1 nM bifunctional fusion protein+TGF-β1, Test 2 represents 10 nM bifunctional fusion protein+TGF-β1, and Test 3 represents 100 nM bifunctional fusion protein+TGF-β1.

FIG. 13 shows experimental results of the tumor growth suppression effect of the bifunctional fusion protein in mice according to Example 11 of the present invention, wherein the curves in each figure represent the tumor growth curves of animals in that treatment group.

### DETAILED DESCRIPTION OF THE INVENTION

To make the objectives, technical solutions, and beneficial technical effects of the present invention clearer, the present invention is further detailed in the following in conjunction with the embodiments, and those skilled in the art can easily understand other advantages and efficacy of the present invention according to the contents disclosed in this specification.

On the basis of a large number of practical research, the inventors of the present invention utilize site-directed mutagenesis to genetically mutate the VEGF and TGF-β receptors in the fusion protein, thereby significantly alleviating peptide bond fragmentation in the protein purification process. This leads to reduced difficulty of purification, and thus the bifunctional fusion protein that is highly stable and able to inhibit VEGF and TGF-β simultaneously can be prepared.

A first aspect of the present invention provides a bifunctional fusion protein, the bifunctional fusion protein comprises an anti-VEGF fragment and an anti-TGF-β fragment. The anti-VEGF fragment in the bifunctional fusion protein is typically a fragment subjected to genetic mutation, for example, it may be a fragment mutated from any existing known anti-VEGF fragment. The anti-VEGF fragment may be a VEGF receptor fragment. The anti-TGF-β fragment may be a TGF-β receptor fragment subjected to mutation, for example, it may be a fragment mutated from any existing known anti-TGF-β fragment.

The anti-VEGF fragment comprises a heavy chain, the heavy chain comprises at least an amino acid mutation at position 453 compared to an original heavy chain.

The anti-TGF-β fragment comprises at least an amino acid mutation at position 8 compared to an original anti-TGF-β fragment.

Preferably, the amino acid mutation at position 453 of the heavy chain of the anti-VEGF fragment comprises an amino acid deletion or substitution. The substitution refers to replacing amino acid residue K at position 453 with one of other non-K amino acids or derivatives thereof, such as replacing K with G, A, V, L, I, P, F, W, M, Y, S, T, C, N, Q, D, E, R, or H amino acid residue.

Preferably, the amino acid mutation at position 8 of the anti-TGF-β fragment comprises an amino acid deletion or substitution. The substitution refers to replacing amino acid residue K at position 8 with other non-K amino acids or derivatives thereof, such as replacing K with G, A, V, L, I, P, F, W, M, Y, S, T, C, N, Q, D, E, R, or H amino acid residue. Preferably, the mutation of amino acid residue at position 8 of the anti-TGF-β fragment is mutating K to A (K→A).

Preferably, the bifunctional fusion protein of the present invention comprises at least one of the foregoing mutations.

Preferably, the amino acid sequence of the original heavy chain is shown as SEQ ID No. 6.

Preferably, the amino acid sequence of the original anti-TGF-β fragment is shown as SEQ ID No. 7.

In some preferred embodiments, the bifunctional fusion protein comprises an amino acid deletion mutation at position 453 and a K-to-A mutation of the amino acid at position 8.

In some preferred embodiments, the heavy chain of the anti-VEGF fragment may specifically comprise:
a-2) a polypeptide fragment with an amino acid sequence shown as SEQ ID No. 2; or,
b-2) a polypeptide fragment with an amino acid sequence having more than 90% sequence identity to SEQ ID No. 2, wherein the polypeptide fragment comprises an amino acid residue deletion mutation at position 453 and has the function of the polypeptide fragment in a-2). Specifically, the polypeptide fragment in b-2) specifically refers to: a polypeptide fragment obtained by substitution, deletion, or addition of one or more (specifically 1-50, 1-30, 1-20, 1-10, 1-5, 1-3, 1, 2, or 3) amino acids in the amino acid sequence shown as SEQ ID No. 2, or by the addition of one or more (specifically 1-50, 1-30, 1-20, 1-10, 1-5, 1-3, 1, 2, or 3) amino acids at the N-terminus and/or C-terminus of the amino acid sequence shown as SEQ ID No. 2, while retaining the function of the polypeptide fragment with the amino acid sequence shown as SEQ ID No. 2. For example, the function may be anti-VEGF. The amino acid sequence in b-2) may have more than 90%, 91%, 92%, 93%, 94%, 95%, 96%, 97%, 98%, or 99% sequence identity to SEQ ID No. 2.

In some preferred embodiments, the anti-TGF-β fragment may specifically comprise:
c) a polypeptide fragment with an amino acid sequence shown as SEQ ID No. 3; or,
d) a polypeptide fragment with an amino acid sequence having more than 90% sequence identity to SEQ ID No. 3, wherein the polypeptide fragment comprises a K8A mutation (mutating K to A) of amino acid residue at position 8 and has the function of the polypeptide fragment in c). Specifically, the polypeptide fragment in d) refers to: a polypeptide fragment obtained by substitution, deletion, or addition of one or more (specifically 1-50, 1-30, 1-20, 1-10, 1-5, 1-3, 1, 2, or 3) amino acids in the amino acid sequence shown as SEQ ID No. 3, or by the addition of one or more (specifically 1-50, 1-30, 1-20, 1-10, 1-5, 1-3, 1, 2, or 3) amino acids at the N-terminus and/or C-terminus of the amino acid sequence shown as SEQ ID No. 3, while retaining the function of the polypeptide fragment with the amino acid sequence shown as SEQ ID No. 3. For example, the function may be anti-TGF-β. The amino acid sequence in d) may have more than 90%, 91%, 92%, 93%, 94%, 95%, 96%, 97%, 98%, or 99% sequence identity to SEQ ID No. 3.

In some preferred embodiments, the anti-VEGF fragment further comprises a light chain. The light chain may specifically comprise:
a-1) a polypeptide fragment with an amino acid sequence shown as SEQ ID No. 1; or,
b-1) a polypeptide fragment with an amino acid sequence having more than 90% sequence identity to SEQ ID No. 1 and the function of the polypeptide fragment in a-1). Specifically, the polypeptide fragment in b-1) refers to: a polypeptide fragment obtained by substitution, deletion, or addition of one or more (specifically 1-50, 1-30, 1-20, 1-10, 1-5, 1-3, 1, 2, or 3) amino acids in the amino acid sequence shown as SEQ ID No. 1, or by the addition of one or more (specifically 1-50, 1-30, 1-20, 1-10, 1-5, 1-3, 1, 2, or 3) amino acids at the N-terminus and/or C-terminus of the amino acid sequence shown as SEQ ID No. 1, while retaining the function of the polypeptide fragment with the amino acid sequence shown as SEQ ID No. 1. For example, the function may be anti-VEGF. The amino acid sequence in b-1) may have more than 90%, 91%, 92%, 93%, 94%, 95%, 96%, 97%, 98%, or 99% sequence identity to SEQ ID No. 1.

In the bifunctional fusion protein provided by the present invention, the bifunctional fusion protein may further comprise a linking peptide fragment. Preferably, the linking peptide fragment is enriched in G, S, and/or A, wherein “enriched in G, S, and/or A” refers to being enriched in G, S, and A, enriched in G and S, or enriched in G and A. More preferably, the linking peptide fragment comprises a polypeptide fragment with an amino acid sequence shown as SEQ ID NO. 5.

In the bifunctional fusion protein provided by the present invention, the bifunctional fusion protein may sequentially comprise, from the N-terminus to the C-terminus, the anti-VEGF fragment and the anti-TGF-β fragment.

In a specific embodiment of the present invention, the bifunctional fusion protein may comprise a heavy chain with an amino acid sequence shown as SEQ ID NO. 4 and a light chain with the amino acid sequence shown as SEQ ID NO. 1.

A second aspect of the present invention provides an isolated polynucleotide encoding the bifunctional fusion protein provided in the first aspect of the present invention.

A third aspect of the present invention provides a construct, wherein the construct comprises the isolated polynucleotide provided in the second aspect of the present invention. Suitable methods for providing the construct are known to those skilled in the art. For example, the construct can typically be obtained by inserting the isolated polynucleotide into a suitable vector.

A fourth aspect of the present invention provides an expression system, wherein the expression system comprises the construct provided in the third aspect of the present invention or a genome incorporating an exogenous polynucleotide provided in the second aspect of the present invention, so as to express the fusion protein. The expression system may usually be a host cell, and the host cell may be one or more of bacteria, fungi, mammalian cells, etc.

The bacteria are selected from one or more of the following: Escherichia coli, Lactobacillus casei, Bacteroides fragilis, Acinetobacter lwoffii, Fusobacterium nucleatum, Bacteroides georgiae, Bacteroides arabidopsis, Lactobacillus rhamnosus, Bacteroides massiliensis, Bacteroides ovatus, Campylobacter jejuni, Staphylococcus saprophyticus, Enterococcus faecium, Bacteroides thetaiotaomicron, Bacteroides vulgatus, Bacteroides uniformis, Parabacteroides merdae, Fusobacterium mortiferum, and Bifidobacterium breve.

The fungi are selected from yeast and/or molds. The yeast is selected from one or more of the following: Saccharomyces cerevisiae, Candida dubliniensis, Candida glabrata, Candida guilliermondii, Candida kefyr, Candida krusei, Hansenula polymorpha, Pichia pastoris, Kluyveromyces fragilis, Kluyveromyces lactis, Schizosaccharomyces pombe, Candida albicans, Candida lusitaniae, Candida melinii, Candida oleophila, Candida parapsilosis, Candida tropicalis, and Candida utilis. The mold is selected from one or more of the following: Aspergillus clavatus, Aspergillus glaucus group, Aspergillus fumigatus, Aspergillus flavus, Aspergillus niger, Aspergillus nidulans, Aspergillus oryzae, Aspergillus terreus, Aspergillus ustus, and Aspergillus versicolor.

The mammalian cell is selected from one or more of the following: CHO cell, HEK 293 human embryonic kidney cell, Bowes melanoma cell, COS-7 cell, C127 cell, HeLa cell, BHK cell, SP2/0 mouse plasma cell, NS0 mouse plasma cell, COS monkey kidney cell, CHO-S, R1 mouse embryonic cell, 3T3 mouse fibroblast, BHK21 Syrian hamster fibroblast, MDCK canine epithelial cell, PtKI Potorous tridactylus epithelial cell, E14.1 mouse embryonic cell, H1 human embryonic cell, H9 human embryonic cell, and PER C.6 human embryonic cell. In some preferred embodiments, the mammalian cell is selected from CHO cell and/or HEK293 human embryonic kidney cell. In some other preferred embodiments, the host cell is a CHO-K1 cell.

A fifth aspect of the present invention provides a method for preparing the bifunctional fusion protein provided in the first aspect of the present invention. Suitable methods for preparing the fusion protein are known to a person skilled in the art. For example, the method for preparing the bifunctional fusion protein may comprise: culturing the expression system provided in the fourth aspect of the present invention under suitable conditions to express the fusion protein, collecting the culture containing the fusion protein, and performing isolation and purification.

A sixth aspect of the present invention provides a use of the bifunctional fusion protein provided in the first aspect of the present invention in the preparation of drugs for the treatment of diseases associated with VEGF and/or TGF-β expression. Preferably, the disease is one or more of tumors, autoimmune diseases, and ophthalmic diseases. The bifunctional fusion protein provided by the present invention not only has good stability, but also exhibits excellent affinity for both TGF-β and VEGF. Compared with the innovator drug Avastin and the control drug M7824, it has a similar binding ability to the antigens. This allows the bifunctional fusion protein to be used in the treatment of diseases associated with the expression of VEGF and/or TGF-β.

The above-described diseases associated with VEGF and/or TGF-β expression can be tumors and the like, specifically solid tumors or hematological tumors, and more specifically colorectal cancer (CRC), hepatocellular carcinoma (HCC), ovarian cancer (OC), cervical carcinoma (CCA), thyroid cancer (TC), melanoma (MM), lung cancer (LC), head and neck cancer (HNC), nasopharyngeal cancer (NPC), glioblastoma (GBM), prostate carcinoma (PCA), pheochromocytoma and paraganglioma (PPGL), gastric cancer (GC), esophageal cancer (EC), renal cell cancer (RCC), bladder uroepithelial carcinoma (BLCA), pancreatic adenocarcinoma (PAC), breast carcinoma (BRCA), lymphoma, sarcoma (SAR), acute or chronic leukemia, etc.

The disease associated with VEGF and/or TGF-β expression may be an autoimmune disease, the autoimmune disease is one or more of systemic lupus erythematosus, rheumatoid arthritis, systemic sclerosis, sjögren's syndrome, and polymyositis.

The disease associated with VEGF and/or TGF-β expression may be an ophthalmic disease, the ophthalmic disease includes, but is not limited to, dry AMD, wet AMD, and choroidal neovascularization (CNV). For example, the ophthalmic disease may be one or more of age-related macular degeneration (AMD), choroidal neovascularization (CNV), choroidal neovascular membrane (CNVM), cystoid macular edema (CME), epiretinal membrane (ERM), and macular hole; myopia-associated CNVM, angioid streaks, retinal detachment, diabetic retinopathy, diabetic macular edema (DME), atrophic or hypertrophic lesions of the retinal pigment epithelium (RPE) cell, retinal vein occlusion, choroidoretinal vein occlusion, and macular edema; corneal neovascularization due to hypoxia, pterygium, subretinal edema, and intraretinal edema; and macular edema due to retinal vein occlusion, retinitis pigmentosa, Stargardt disease, glaucoma, inflammatory diseases, cataracts, refractory anomalies, keratoconus, retinopathy of prematurity, anterior segment neovascularization, corneal neovascularization post-keratitis, corneal transplantation, and keratoplasty.

A seventh aspect of the present invention provides a pharmaceutical composition comprising the bifunctional fusion protein provided in the first aspect of the present invention or the expression system provided in the fourth aspect of the present invention or the culture of the expression system. The pharmaceutical composition may further comprise a pharmaceutically acceptable carrier. The carrier may include a variety of excipients and diluents. The carrier itself is not a necessary active ingredient and does not exhibit excessive toxicity upon administration. Suitable carriers are well known to those skilled in the art, for example, a full discussion of pharmaceutically acceptable carriers can be found in Remington's Pharmaceutical Sciences (Mack Pub. Co., N.J., 1991).

In the present invention, the bifunctional fusion protein provided in the first aspect of the present invention can be used as a single active ingredient or in combination with other active components in the above-described drugs or compositions.

An eighth aspect of the present invention provides a treatment method, wherein the method comprises: administering to an individual a therapeutically effective amount of the bifunctional fusion protein provided in the first aspect of the present invention or the composition provided in the seventh aspect of the present invention. The method provided by the present invention can be used for the treatment of the disease associated with VEGF and/or TGF-β expression, which can specifically be a tumor, etc.

The bifunctional fusion protein in the antibody, use, or pharmaceutical composition described herein can also be used in combination with other cancer treatment methods or drugs (including standard treatment methods/means for tumors), including, but not limited to, surgery, radiotherapy, chemotherapy, tumor immunotherapy, and the like. The bifunctional fusion protein, when used in combination with other drugs for tumor therapy, can be prepared into the pharmaceutical composition or administered separately from other drugs for tumor therapy. When the bifunctional fusion protein described herein is prepared into the pharmaceutical composition, the active ingredient of the pharmaceutical composition further comprises other drugs for tumor therapy, and the mass ratio of the bifunctional fusion protein to other drugs for tumor therapy may be any proper ratio such as 1:(1-99), specifically for example 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. The mass ratio of other drugs for tumor therapy to the bifunctional fusion protein may be any proper ratio such as 1:(1-99), specifically for example 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1.57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. The separate administration means that the bifunctional fusion antibody described herein can be administered before, simultaneously with, or after other drugs for tumor therapy.

For tumor radiotherapy, common types of radiation include, but are not limited to, X-rays, gamma rays, and charged particles such as electrons, protons, and heavy ions.

For tumor chemotherapy, common chemotherapeutic agents include: \textcircled{1} cytotoxic agents: alkylating agents such as nitrogen mustard, carmustine (BCNU), cyclophosphamide, busulfan (Myleran), and lomustine (CCNU); \textcircled{2} antimetabolites: such as fluorouracil, methotrexate, cytarabine, mercaptopurine, and tegafur (ftorafur); and \textcircled{3} antibiotics: such as actinomycin D (dactinomycin), mitomycin, bleomycin, adriamycin, bleomycin A5, daunorubicin, plicamycin, etc.; \textcircled{4} alkaloids: such as vincristine, vinblastine, hydroxycamptothecin, and podophyllotoxin like etoposide (VP-16) and teniposide (VM-26); \textcircled{5} (hormones, such as tamoxifen, diethylstilbestrol, progesterone, testosterone propionate, thyroxine, prednisone, dexamethasone, etc.; \textcircled{6} other: such as procarbazine, hydroxyurea, L-asparaginase, cisplatin, carboplatin, antimony anti-cancer agent, dacarbazine, and so on.

For tumor immunotherapy, it mainly includes immunomodulators, adoptive cell transfer therapy (ACT), tumor-specific vaccines, and small molecule immune drugs.

The immunomodulators include activators of co-stimulatory molecules, immune checkpoint inhibitors (ICBs), immune checkpoint activators, and the like.

In some embodiments, the immunomodulator is an activator of a co-stimulatory molecule. In one embodiment, the activator of the co-stimulatory molecule is selected from activators (e.g., an agonistic antibody, or antigen-binding fragment or a soluble fusion protein thereof) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, and CD83 ligand.

In some embodiments, the immunomodulator is an inhibitor or activator of an immune checkpoint molecule, and the immune checkpoint molecule is involved in the immune checkpoint stimulatory pathway and inhibitory pathway. Immune checkpoint inhibitors are monoclonal antibody drugs that target the corresponding immune checkpoints, and their main function is to block the interaction between tumor cells expressing immune checkpoints and immune cells, thereby preventing the suppression of immune cells by tumor cells and affecting the immune response. In one embodiment, the monoclonal antibody molecule is a full antibody or a fragment thereof (e.g., Fab, F(ab′)₂, Fv, and a single-chain Fv fragment (scFv)). In other embodiments, the monoclonal antibody molecule has a heavy chain constant region (Fc) selected from, for example, the heavy chain constant regions of IgG1, IgG2, IgG3, IgG4, IgM, IgA1, IgA2, IgD, and IgE. In one embodiment, the constant region is altered (e.g., mutated) to modify antibody molecular property (e.g., to increase or decrease one or more of the following: Fc receptor binding, antibody glycosylation, number of cysteine residues, effector cell function, or complement function).

In one embodiment, the immune checkpoint inhibitor is an inhibitor of PD-1, PD-L1, PD-L2, PD-L3, CTLA4, TIM-3, LAG3, CEACAM (e.g., CEACAM-1, CEACAM-3, and/or CEACAM-5), VISTA, VSIR, BTLA, TIGIT, LAIR1, LMTK3, TIGHT, IDO, CD27L, CD40, CD47, CD137, CD160, CD244, CD270, GITR, B7-H1, B7-1, 2B4 and/or TGF-β R. In one embodiment, the immune checkpoint inhibitor can inhibit PD-1, PD-L1, PD-L2, PD-L3, CTLA4, TIM-3, LAG3, CEACAM (e.g., CEACAM-1, CEACAM-3 and/or CEACAM-5), VISTA, VSIR, BTLA, TIGIT, LAIR1, LMTK3, TIGHT, IDO, CD27L, CD40, CD47, CD137, CD160, CD244, CD270, GITR, B7-H1, B7-1, 2B4 and/or TGFRβ, or any combination thereof. In another embodiment, the immune checkpoint inhibitor can inhibit PD-1, PD-L1, LAG-3, TIM-3, CEACAM (e.g., CEACAM-1, -3, and/or -5), or CTLA4, or any combination thereof. In another embodiment, the bifunctional fusion protein can be used in combination with an immune checkpoint inhibitor that inhibits PD-1 and/or PD-L1. The term “inhibit” or “inhibitor” refers to reducing the activity of a specified molecule, e.g., PD-1 or PD-L1, by at least 5%, 10%, 20%, 30%, 40%, or more. Thus, the inhibition does not necessarily need to be 100%.

The adoptive cell transfer therapy (ACT) comprises tumor-infiltrating lymphocyte (TIL) therapy, engineered T-cell receptor (TCR) therapy, chimeric antigen receptor T-cell (CART) therapy, and NK cell therapy.

The tumor-specific vaccines comprise whole tumor cell vaccines, dendritic cell vaccines, gene vaccines, RNA vaccines, and protein peptide vaccines.

Whole tumor cell vaccines are created by treating autologous or allogeneic tumor cells through physical, chemical, biological, or other methods to render them non-tumorigenic while retaining their antigenicity. These vaccines can be combined with nonspecific stimulatory factors (such as the BCG vaccine) to actively immunize cancer patients.

Dendritic cell vaccines: dendritic cells are a group of heterogeneous immune cells with the strongest antigen-presenting capability and are the only professional antigen-presenting cells capable of activating naive T cells.

Genetic vaccines are products obtained by cloning the gene fragment encoding the protective antigen of a pathogen into an expression vector and then using the vector to transfect cells, eukaryotic microorganisms, or prokaryotic microorganisms. Alternatively, gene-deficient vaccines without virulence-related genes can be obtained by deleting the virulence-related gene of a pathogen, which mainly include viral and bacterial vectors.

Many viruses can be used as vectors for recombinant vaccines, such as adenovirus, poxvirus, etc., and bacterial vectors mainly used are Listeria monocytogenes and Salmonella monocytogenes.

Protein peptide vaccines are prepared by chemical synthesis techniques based on the amino acid sequence of a known or predicted antigenic epitope in the antigenic gene of a pathogen. If a specific antigen in tumor cells can be found and a corresponding cancer vaccine can be developed based on this amino acid sequence, the relevant immune cells will be activated to kill the tumor cells with the same specific antigen; e.g., EGF vaccine (Cimavax), MAGE-A3, TG4010, etc.

In the present invention, sequence identity refers to the percentage of identical residues in the sequences being compared. Sequence identity of two or more target sequences may be calculated using computational software well known in the art from NCBI, etc.

In the present invention, the term “individual” typically includes humans, non-human primates, and other mammals such as rodents, dogs, cats, horses, sheep, pigs, cows, etc., which may benefit from treatment utilizing the aforementioned drugs, compositions, formulations, kits, or combination formulations.

In the present invention, “therapeutically effective amount” generally refers to a dose which, after a suitable period of administration, is capable of achieving a therapeutic effect for the disease such as those listed above.

The bifunctional fusion protein provided by the present invention can antagonize VEGF and TGF-β simultaneously with high efficiency. The stability of the modified protein has been significantly improved, therefore, it is less prone to fragmentation during the purification process. This significantly reduces the difficulty of purifying the protein while guaranteeing the potency of the drug, thus improving the efficiency of the purification and reducing the production cost. In addition, the VEGF/TGF-β bifunctional fusion protein provided by the present invention also has the advantages of high potency and low side effects, having good industrialization prospects.

The following examples further illustrate the present invention, but do not limit the scope of the present invention.

Unless otherwise specified, the experimental methods, detection methods, and preparation methods disclosed in the present invention are conventional in the fields of molecular biology, biochemistry, chromatin structure and analysis, analytical chemistry, cell culture, recombinant DNA technology, etc. These techniques are well described in the existing literature, see Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119. Chromatin Protocols (P. B. Becker, ed.) Humana Press, Totowa, 1999, etc.

### Example 1 Preparation of Bifunctional Fusion Protein

Polynucleotides encoding the heavy chain (SEQ ID NO. 4) and the light chain (SEQ ID NO. 1) of a bifunctional fusion protein were co-transfected into CHO-K1 expression host cells using the same expression vector, high-expression clones were screened using methionine sulfoximine (MSX) under selective pressure and then incubated in EmCD CHO 104 medium (Eminence) at 37° C. and 5% CO₂ for mass culture and induction of expression, and the cell culture fluid was collected after 12-14 days.

### Example 2 Purification of Bifunctional Fusion Protein

The cell culture fluid collected in Example 1 was subjected to centrifugation and then high-speed centrifugation, followed by affinity chromatography purification. Unimab 50HC purification packing and AKTA (GE healthcare) protein purification system from NanoMicro Technology were used. Samples were loaded at a concentration of 30 mg protein/ml media, the equilibration buffer was 50 mM TrisHCl+150 mM NaCl solution at pH 7.5, and the elution buffer was 20 mM Na-Citrater solution at pH 3.0, with a flow rate of 5 ml/min. The eluted fraction containing the target protein was collected, and then the pH was adjusted to 7.0 for the subsequent detection and characterization of physicochemical properties and biological activities.

### Example 3 Preparation and Purification of Control Fusion Protein

The amino acid sequence of the control fusion protein includes a heavy chain with an amino acid sequence shown as SEQ ID NO. 8 and a light chain with an amino acid sequence shown as SEQ ID NO. 7.

The preparation process is the same as that in Example 1, and the purification process is the same as that in Example 2.

### Example 4 SDS-PAGE Electrophoresis Identification of Bifunctional Fusion Protein

### 4.1 Determination of Molecular Weight and Purity of Bifunctional Fusion Protein by SDS-PAGE Electrophoresis

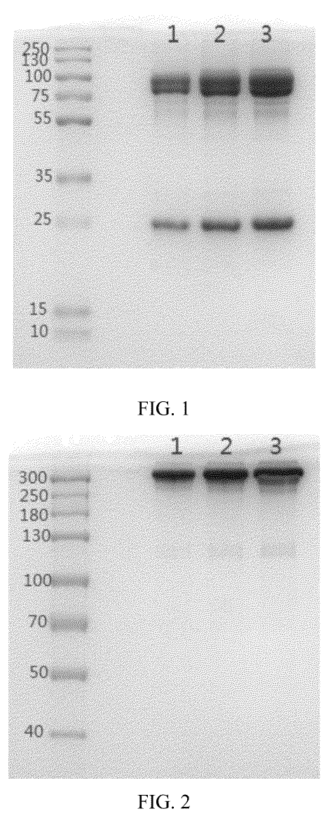

The bifunctional fusion protein obtained in Example 2 was subjected to SDS-PAGE electrophoresis under reducing (β-mercaptoethanol) and non-reducing conditions. Polyacrylamide gels with concentrations of 12% and 8% were used for separation, with a sample loading of 3 μg protein per well. The electrophoresis results are shown in FIG. 1 (reducing SDS-PAGE) and FIG. 2 (non-reducing SDS-PAGE). Lanes 1, 2, and 3 correspond to three replicates of the bifunctional fusion protein targeting VEGF and TGF-β. The electrophoresis results indicate that the target protein has a molecular weight of approximately 179 kD and a purity greater than 98%.

### 4.2 Determination of Stability of Bifunctional Fusion Protein by SDS-PAGE Electrophoresis

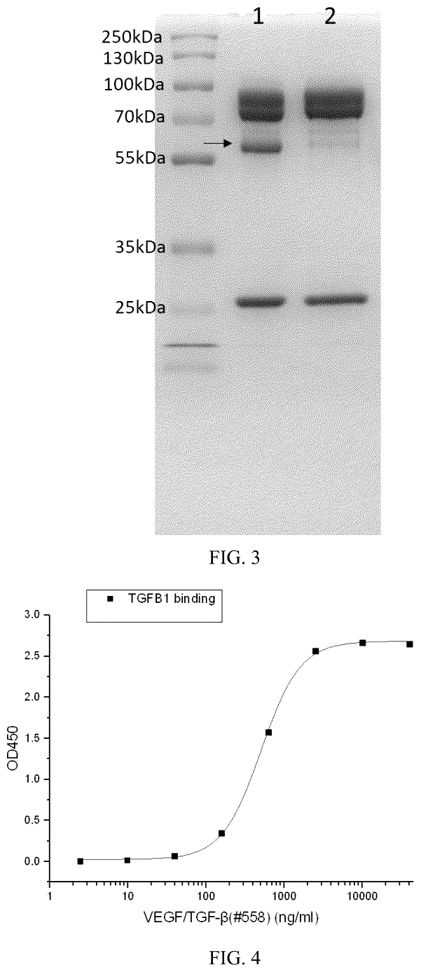

The bifunctional fusion protein obtained in Example 2 and the control fusion protein obtained in Example 3 were characterized by SDS-PAGE electrophoresis under reducing (β-mercaptoethanol) conditions. Polyacrylamide gels with concentrations of 12% and 8% were used for separation, with a sample loading of 3 μg protein per well. The electrophoresis results are shown in FIG. 3 (reducing SDS-PAGE). Lanes 1 and 2 correspond to the control fusion protein obtained in Example 3 and the fusion protein obtained in Example 2, respectively. As can be seen from FIG. 3, the control fusion protein, i.e., the protein without the K→A mutation, is prone to fragmentation (obvious fragmented proteins are present at the place marked with an arrow), however, the bifunctional fusion protein containing the K→A mutation has no obvious fragmented proteins present at the place corresponding to the arrow in lane 1. This indicates that the bifunctional fusion protein obtained in Example 2 of the present invention has a very high stability.

### Example 5 ELISA Assay of Recombinant Human TGF-β 1/2/3 Binding

ELISA assay was performed following standard procedures:
5.1 Recombinant human TGF-β 1, TGF-β 2, or TGF-β 3 dissolved in a solution containing 15 mmol/L Na₂CO₃, 35 mmol/L NaHCO₃, and 7.7 mmol/L NaN₃ at pH 9.6 was coated on a 96-well plate at room temperature overnight, with each well coated with 0.5 μg.
5.2 The next day, the 96-well plate was rinsed four times using a TBS solution containing 0.05% Tween-20 at pH 7.4. The plate was blocked using a TBS solution containing 0.05% Tween-20 and 5% non-fat milk powder at pH 7.4 for 1 hour at room temperature. After removing the blocking solution, different concentrations of bifunctional fusion proteins or control drug M7824 were added to the wells in duplicates, and the plate was incubated at room temperature for 2 hours.
5.3 Subsequently, the plate was washed four times using a washing solution, followed by the addition of anti-human IgG (Fc-specific) peroxidase-conjugated goat antibody and incubation in the dark at room temperature for 30 minutes. The color reaction was then stopped using 1M H₂SO₄.
5.4 The absorbance was read at 450 nm using an Envision reader, with correction at 570 nm. Detailed results are shown in FIGS. 4, 5, and 6.

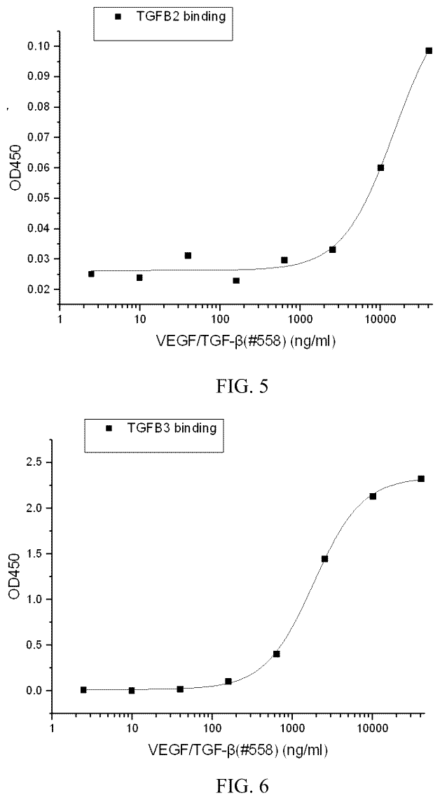

As can be seen from FIG. 4, FIG. 5, and FIG. 6, the affinity of the bifunctional fusion protein obtained in Example 1 for TGF-β is basically comparable to that of M7824, both showing weak affinity for TGF-β2. The EC50 values for the affinity of the bifunctional fusion protein obtained in Example 1 and M7824 for TGF-β1 are 2.843 nM and 2.076 nM, respectively, and the EC50 values for the affinity for TGF-β3 are 10.309 nM and 3.323 nM, respectively.

### Example 6 ELISA Assay of Recombinant Human VEGF Binding

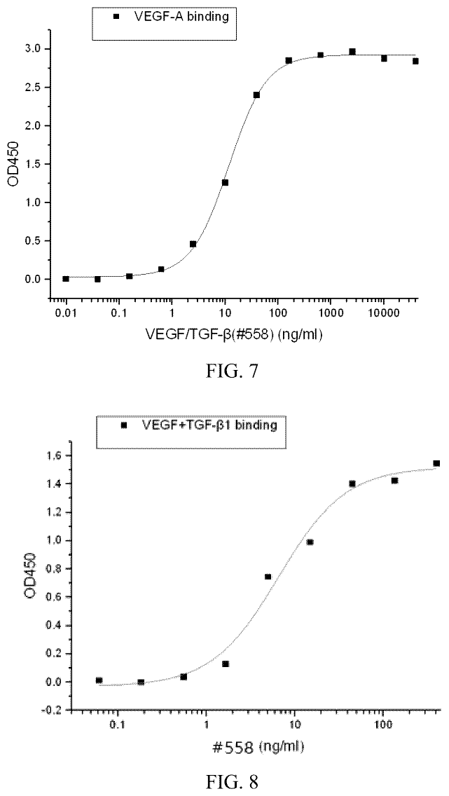

To test whether the bifunctional fusion protein prepared in Example 1 binds to human VEGF, recombinant human VEGF (Novoprotein) was coated on a 96-well plate overnight at room temperature, with each well coated with 0.5 g. Experiments were performed on the bifunctional fusion protein prepared in Example 1 or the control drug Avastin, experimental methods and steps were the same as those in Example 3, and the specific results are shown in FIG. 7.

As can be seen from FIG. 7, the affinity of the bifunctional fusion protein obtained in Example 1 for VEGF is basically comparable to that of Avastin, and the EC50 values for the affinity for VEGF are 0.0659 nM and 0.0168 nM, respectively.

### Example 7 ELISA Assay of Simultaneous Binding of TGF-β and VEGF

To test whether the bifunctional fusion protein prepared in Example 1 binds to recombinant human TGF-β1/2/3 and VEGF at the same time, recombinant human TGF-β1/2/3 and VEGF were coated on a 96-well plate overnight at room temperature, with each well coated with 0.5 g of TGF-β1/2/3 and VEGF, respectively. Experiments were performed on the bifunctional fusion protein prepared in Example 1 or the control drug M7824 and Avastin, experimental methods and steps were the same as those in Example 3, and the specific results are shown in FIG. 8.

As can be seen from FIG. 8, the bifunctional fusion protein obtained in Example 1 can simultaneously bind to recombinant human TGF-β and VEGF with an EC50 value of 0.0365 nM. This indicates that the bifunctional fusion protein of the present invention can simultaneously bind to recombinant human TGF-β and VEGF, realizing the inhibition of the interaction between VEGF and its receptor as well as the suppression of the regulatory effect of TGF-β on the immune system. Consequently, the bifunctional fusion protein can effectively inhibit the biological effects of VEGF and TGF-β, including effects on vascular permeability, proliferation, and endothelial cell migration and survival. Additionally, it enhances the production and function of effector T cells and DC cells, etc. thereby inhibiting tumor growth and achieving anti-tumor effects.

### Example 8 Inhibition of VEGF-Induced Human Umbilical Vein Endothelial Cell (HUVEC) Proliferation by Bifunctional Fusion Protein

HUVEC cells were resuspended in a complete medium, diluted, inoculated in a 96-well plate with 5000 cells/well, and incubated overnight in a 37° C., 5% CO₂, high humidity incubator. The next day, the original medium was discarded and ECM+1% FBS medium was added to starve the cells for 24 h. Avastin, the bifunctional fusion protein prepared in Example 1, and HSP088-01 (the isotype control protein of the bifunctional fusion protein prepared in Example 1 that retains the activity of the TGF-β fragment only), after being diluted in a 3-fold gradient from an initial concentration of 10 nM, were added to the wells of the plate containing the cells, followed by incubation for 72 hours at 37° C. in a 5% CO₂ and high-humidity incubator. The 96-well plate was then added with freshly prepared CCK-8 (10%) assay solution and incubated in the incubator at 37° C. for 2~4 h. The absorbance at 450 nm was measured on a SpectraMax M5 device after gentle shaking, and the inhibition rate was calculated using the absorbance at 650 nm as a reference, wherein inhibition rate %=[1−(OD_{drug+VEGF}−OD_{No VEGF})/(OD_{only VEGF}−OD_{No VEGF})]*100). The curves were fitted and the IC₅₀ value was calculated using Graphpad Prism 6 software, and the results are shown in FIG. 9.

The results show that both Avastin and the bifunctional fusion protein obtained in Example 1 can effectively inhibit VEGF-induced HUVEC cell proliferation, with IC₅₀ values of 0.3869 nM and 0.1964 nM, respectively.

### Example 9 Inhibition of TGF-β-Induced Human Breast Cancer MDA-MB-231 Cell Migration by Bifunctional Fusion Protein

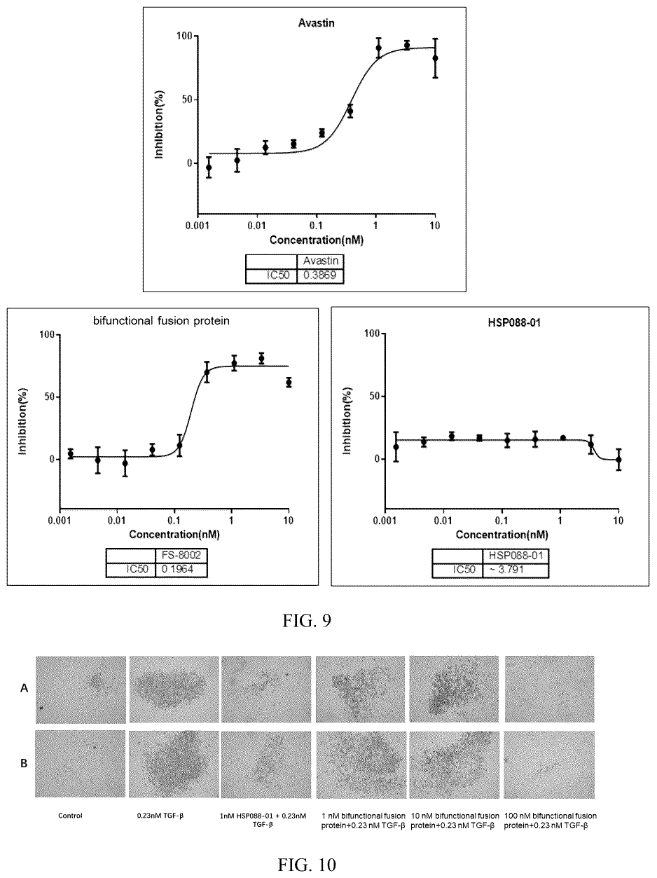

MDA-MB-231 cells were cultured in a complete medium for passaging, and cells in the logarithmic growth phase were collected. After the cells were adhered to the wall, L15+1% FBS medium was added to starve the cells for 16 hours. Matrigel was diluted in serum-free L15 medium at a 1:2 ratio and added to Transwell chambers, incubating at 37° C. for 30 minutes. The starved MDA-MB-231 cells were resuspended in serum-free L15 medium, and then added to each Matrigel-containing Transwell chamber. Blank medium, the bifunctional fusion protein prepared in Example 1, and HSP088-01 (an isotype control protein of the bifunctional fusion protein prepared in Example 1 that retains only TGF-β fragment activity) were added to the chambers. After co-incubating for 1 hour, TGF-β stock solution was added to the upper chamber to a final concentration of 0.23 nM (the blank medium group received the same volume of medium). The total volume of the chambers was 400 μL (including 200 L Matrigel), and the lower chamber contained 600 μL fresh L-15 medium, incubated at 37° C. for 24 hours. After incubation, the cells were fixed with paraformaldehyde for 20 minutes and stained with crystal violet for 20 minutes. After washing with PBS, images were captured using an inverted microscope, and the number of cells was statistically analyzed with Image J software. The results are shown in FIGS. 10 and 11.

The results show that TGF-β is able to induce MDA-MB-231 cell migration, and the number of migrated MDA-MB-231 cells was significantly reduced after the addition of the bifunctional fusion protein prepared in Example 1 and HSP088-01, and the inhibition effect on MDA-MB-231 cell migration was gradually enhanced with the increase of the concentration of the bifunctional fusion protein prepared in Example 1. The inhibitory effect of 100 nM of FS8002 on MDA-MB-231 cell migration was significantly different from that of the negative control containing only TGF-β (p<0.05**).

### Example 10 Neutralization of TGF-β by Bifunctional Fusion Protein

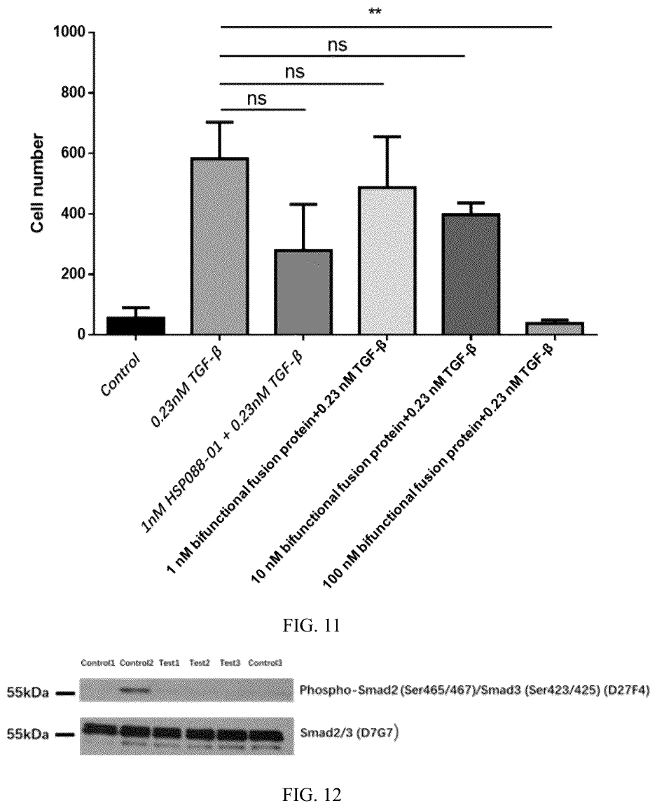

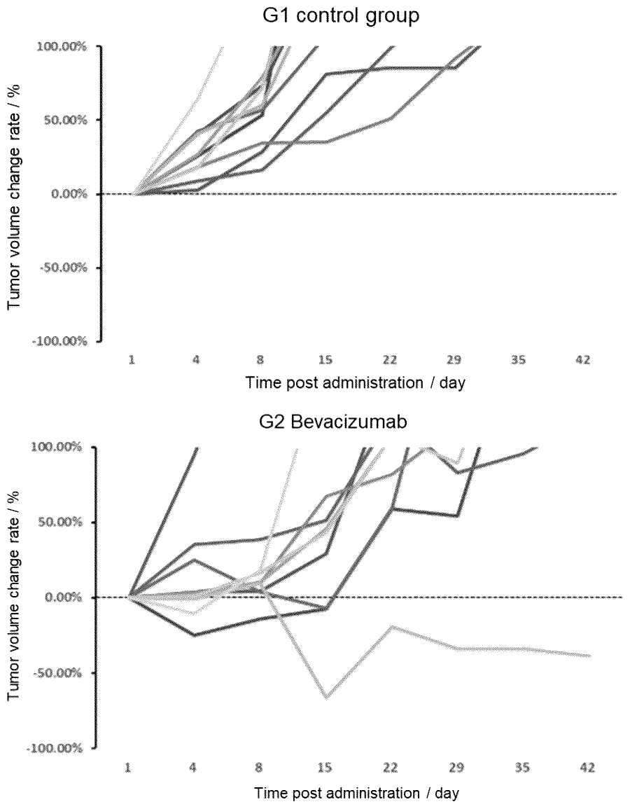

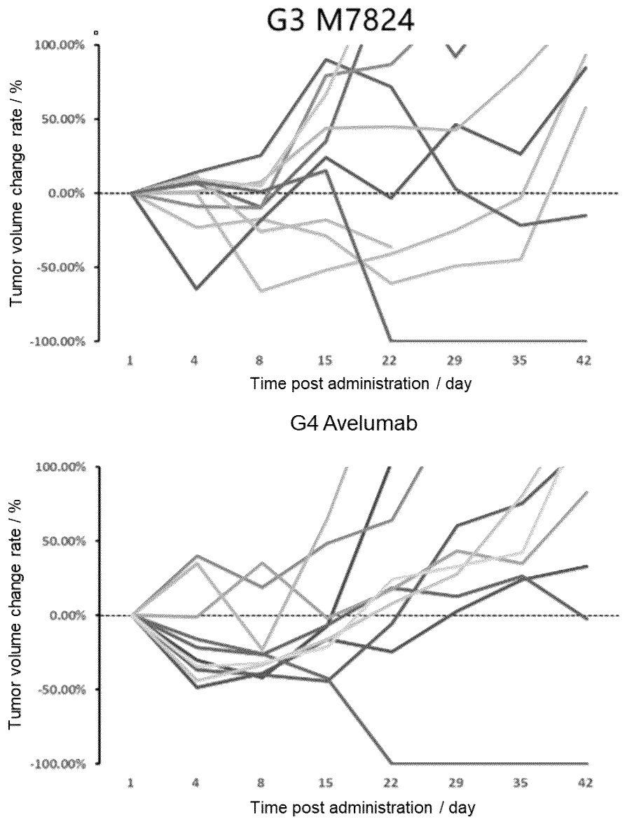

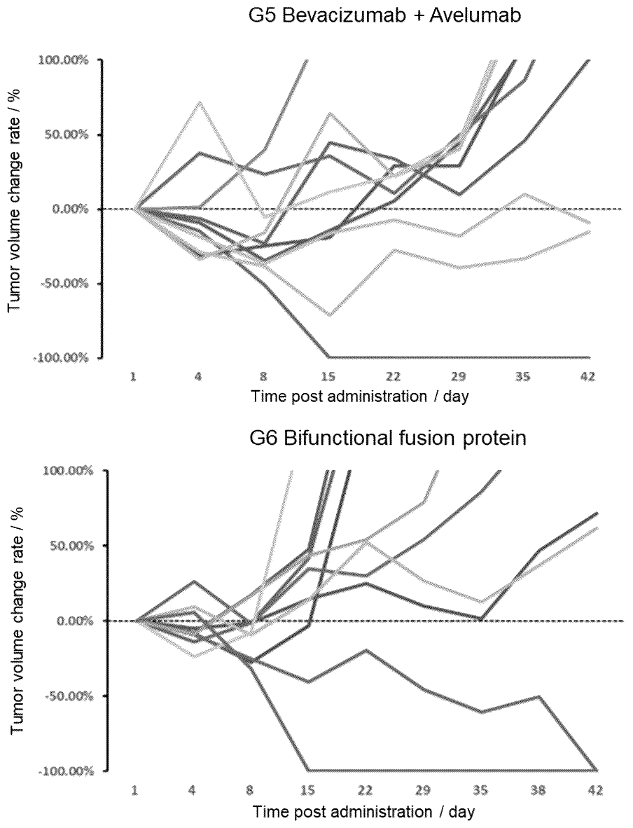

MDA-MB-231 cells were inoculated in a 6-well plate (1×10⁶ cells/well) and cultured at 37° C. for 24 h. After incubation, the medium in different wells was replaced with PBS, PBS+TGF-β1, medium containing TGF-β1 and the bifunctional fusion proteins prepared in Example 1 with different concentrations, or medium containing TGF-β1 and HSP088-01 (an isotype control protein of the bifunctional fusion protein prepared in Example 1 that retains TGF-β fragment activity only), respectively. After the medium replacement, incubation continued for 1 hour. The supernatant was removed from the 6-well plate, and the cells in the wells were washed with D-PBS and then lysed using a lysis buffer. The lysates were immediately subjected to SDS-PAGE electrophoresis, and Western Blot was used to detect Smad2/3 and phosphorylated Smad2/3 (p-Smad2/3) signals. The experimental results are shown in FIG. 12.

The results show that when MDA-MB-231 cells are treated with TGF-β1 only, p-Smad2/3 is detected by Western Blot, indicating that TGF-β1 can induce the classical intracellular Smad signaling pathway by binding to the TGF-β receptor (TGF-βR) on MDA-MB-231 cells. When TGF-β1 and the bifunctional fusion protein obtained in Example 1 with different concentrations or HSP088-01 are added simultaneously, pSmad2/3 is not detected by Western Blot, indicating that both the bifunctional fusion protein obtained in Example 1 and HSP088-01 can neutralize TGF-β1 and block TGF-β1 from binding to the TGF-βR on the MDA-MB-231 cells, thus blocking Smad downstream signaling. Moreover, the bifunctional fusion protein obtained in Example 1 can effectively neutralize TGF-β1 even at a concentration as low as 1 nM.

### Example 11 In Vivo Anti-Tumor Efficacy in Mice by Bifunctional Fusion Protein

MDA-MB-231 cells resuspended in PBS at a concentration of 1×10⁷ cells/0.1 mL were injected subcutaneously into the right flank of BALB/c nude mice, with 0.1 mL (containing 30% Matrigel) per mouse. A total of 100 mice were used.

After 22 days of tumor formation, mice that did not develop tumors and those with the largest and smallest tumor volumes were excluded. Seventy mice with moderate tumor volumes, averaging around 70 mm³, were selected, with 10 mice per group. The drug was administered on the day of grouping, and the bifunctional fusion protein prepared in Example 1 or the control drug was administered twice a week for a total of four weeks.

Tumor volume and body weight of the tumor-bearing mice were measured and recorded during the experiment.

The groups were as follows:
group 1 (G1): control group (saline only);
group 2 (G2): control drug bevacizumab only;
group 3 (G3): control drug M7824 only;
group 4 (G4): control drug avelumab only;
group 5 (G5): control drug bevacizumab in combination with avelumab;
group 6 (G6): the bifunctional fusion protein obtained in Example 1 only;
group 7 (G7): the bifunctional fusion protein obtained in Example 1+avelumab.

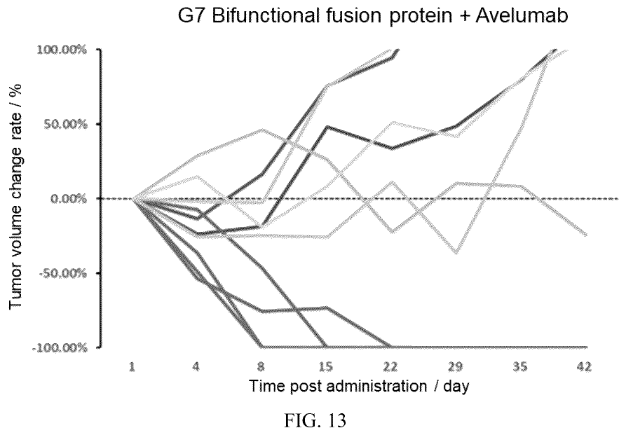

The results are shown in FIG. 13, wherein Spider Plot is performed with the drug administration time as the horizontal coordinate and the change rate of tumor volume as the vertical coordinate. It can be concluded that, compared to G1, G6 (the bifunctional fusion protein obtained in Example 1 only) and G7 (the bifunctional fusion protein obtained in Example 1+avelumab) exhibit significant inhibitory effects on the average tumor volume of the mice. Compared to G2 (control drug bevacizumab only), G6 (the bifunctional fusion protein obtained in Example 1 only) shows a significantly improved inhibitory effect on the tumor volume of mice. Compared with G5 (control drug bevacizumab in combination with avelumab), G7 (the bifunctional fusion protein obtained in Example 1+avelumab) has significantly improved inhibitory effect on the tumor volume of the mice, and even the tumors in four mice have been eliminated after the administration of the drug in G7. Therefore, the animal experiment in this example demonstrates the good inhibitory effect of the bifunctional fusion protein obtained in Example 1 on MDA-MB-231 tumor cells in BALB/c nude mice, illustrating the great potential when the bifunctional fusion protein is combined with other immune checkpoint inhibitors.

In summary, the present invention effectively overcomes the drawbacks of the prior art and has highly industrialization and commercial value.

Sequence information:

SEQ ID No. 1
Light chain:
DIQMTQSPSSLSASVGDRVTITCSASQDISNYLNWYQQKPGKAPKVLIYFTS
SLHSGVPSRFSGSGSGTDFTLTISSLQPEDFATYYCQQYSTVPWTFGQGTKVEIKRT
VAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID No. 2
Heavy chain:
EVQLVESGGGLVQPGGSLRLSCAASGYTFTNYGMNWVRQAPGKGLEWV
GWINTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSV
MHEALHNHYTQKSLSLSPG
SEQ ID No. 3
GIPPHVQASVNNDMIVTDNNGAVKFPQLCKFCDVRFSTCDNQKSCMSNCS
ITSICEKPQEVCVAVWRKNDENITLETVCHDPKLPYHDFILEDAASPKCIMKEKKK
PGETFFMCSCSSDECNDNIIFSEEYNTSNPD
SEQ ID No. 4
Heavy chain:
EVQLVESGGGLVQPGGSLRLSCAASGYTFTNYGMNWVRQAPGKGLEWV
GWINTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSV
MHEALHNHYTQKSLSLSPGAGGGGSGGGGSGGGGSGGGGSGIPPHVQASVNND
MIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDN
IIFSEEYNTSNPD
SEQ ID No. 5
AGGGGSGGGGSGGGGSGGGGS
SEQ ID No. 6
Heavy chain:
EVQLVESGGGLVQPGGSLRLSCAASGYTFTNYGMNWVRQAPGKGLEWV
GWINTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGF
YPSDIA VEWESNGQPENNYKTTPPVLDSDGSFFLYSKLTVDKSRWQQGNVFSCSV
MHEALHNHYTQKSLSLSPGK
SEQ ID No. 7
Heavy chain:
GIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCIMKEKKK
PGETFFMCSCSSDECNDNIIFSEEYNTSNPD
SEQ ID No. 8
Heavy chain:
EVQLVESGGGLVQPGGSLRLSCAASGYTFTNYGMNWVRQAPGKGLEWV
GWINTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSV
MHEALHNHYTQKSLSLSPGKAGGGGSGGGGSGGGGSGGGGSGIPPHVQKSVNND
MIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDN
IIFSEEYNTSNPD

The foregoing embodiments are merely illustrative of the principles and potency/efficacy of the present invention and are not intended to limit the present invention. Any person skilled in the art may modify or change the above embodiments without violating the spirit and scope of the present invention. Therefore, all equivalent modifications or changes made by persons having ordinary knowledge in the art without departing from the spirit and technical ideas disclosed in the present invention shall be still covered by the claims of the present invention.

1. A bifunctional fusion protein, comprising an anti-VEGF fragment and an anti-TGF-β fragment;
wherein the anti-VEGF fragment comprises a heavy chain, the heavy chain comprises an amino acid mutation at least at position 453 compared to an original heavy chain; and/or, the anti-TGF-β fragment comprises an amino acid mutation at least at position 8 compared to an original anti-TGF-β fragment.

2. The bifunctional fusion protein of claim 1, wherein the amino acid sequence of the original heavy chain is shown as SEQ ID No. 6; and/or, the amino acid sequence of the original anti-TGF-β fragment is shown as SEQ ID No. 7.

3. The bifunctional fusion protein of claim 1, wherein the amino acid mutation at least at position 453 comprises an amino acid deletion or substitution; and/or, the amino acid mutation at least at position 8 comprises an amino acid deletion or substitution.

4. The bifunctional fusion protein of claim 3, wherein the amino acid mutation at least at position 453 is an amino acid deletion, and the amino acid mutation at least at position 8 is a K→A mutation.

5. The bifunctional fusion protein of claim 4, wherein the heavy chain of the anti-VEGF fragment comprises:
a-1) a polypeptide fragment with an amino acid sequence shown as SEQ ID No. 2; or,
b-1) a polypeptide fragment with an amino acid sequence having more than 90% sequence identity to SEQ ID No. 2, including a deletion mutation at position 453 and having the function of the polypeptide fragment in a-1).

6. The bifunctional fusion protein of claim 4, wherein the anti-TGF-β fragment comprises:
c) a polypeptide fragment with an amino acid sequence shown as SEQ ID No. 3; or,
d) a polypeptide fragment with an amino acid sequence having more than 90% sequence identity to SEQ ID No. 3, including a K→A mutation at position 8 and having the function of the polypeptide fragment in c).

7. The bifunctional fusion protein of claim 1, wherein the anti-VEGF fragment comprises a light chain, and the light chain comprises:
a-1) a polypeptide fragment with an amino acid sequence shown as SEQ ID No. 1; or,
b-1) a polypeptide fragment with an amino acid sequence having more than 90% sequence identity to SEQ ID No. 1, and having the function of the polypeptide fragment in a-1).

8. The bifunctional fusion protein of claim 1, further comprising a linking peptide fragment; preferably, the linking peptide fragment is enriched in G, S, and/or A; more preferably, the linking peptide fragment comprises a polypeptide fragment with an amino acid sequence shown as SEQ ID NO. 5.

9. The bifunctional fusion protein of claim 1, comprising, in sequence from N-terminus to C-terminus, the anti-VEGF fragment and the anti-TGF-β fragment.

10. The bifunctional fusion protein of claim 1, comprising a heavy chain with an amino acid sequence shown as SEQ ID NO. 4 and a light chain with an amino acid sequence shown as SEQ ID NO. 1.

11. An isolated polynucleotide, encoding the bifunctional fusion protein of claim 1.

12. A construct, comprising the isolated polynucleotide of claim 11.

13. An expression system, comprising a construct or a genome incorporating an exogenous polynucleotide, wherein the construct comprises the polynucleotide, and the polynucleotide encodes the bifunctional fusion protein of claim 1.

14. A method for preparing the bifunctional fusion protein of claim 1, comprising: culturing an expression system under suitable conditions to express the bifunctional fusion protein, and performing isolation and purification to provide the bifunctional fusion protein;
wherein the expression system comprises a construct or a genome incorporating an exogenous polynucleotide, wherein the construct comprises the polynucleotide, and the polynucleotide encodes the bifunctional fusion protein of claim 1.

15. A use of the bifunctional fusion protein of claim 1 in the preparation of drugs.

16. The use of claim 15, wherein the drugs are for the treatment of a disease associated with VEGF expression and/or TGF-β expression; preferably, the disease is one or more of a tumor, an autoimmune disease, and an ophthalmic disease.

17. The use of claim 15, wherein the bifunctional fusion protein is combined with other tumor treatment methods, the other tumor treatment methods comprise surgery, radiotherapy, chemotherapy, and tumor immunotherapy.

18. The use of claim 17, wherein the type of radiation of the radiotherapy comprises X-rays, gamma rays, and charged particles;
wherein drugs suitable for the chemotherapy is one or more of \textcircled{1} cytotoxic agents: nitrogen mustard, carmustine, cyclophosphamide, busulfan, and lomustine; \textcircled{2} antimetabolites: fluorouracil, methotrexate, cytarabine, mercaptopurine, and tegafur; \textcircled{3} antibiotics: actinomycin D, mitomycin, bleomycin, adriamycin, bleomycin A5, daunorubicin, and plicamycin; \textcircled{4} alkaloids: vincristine, vinblastine, hydroxycamptothecin, and podophyllotoxin like etoposide and teniposide; \textcircled{5} hormones: tamoxifen, diethylstilbestrol, progesterone, testosterone propionate, thyroxine, prednisone, and dexamethasone; \textcircled{6} other: procarbazine, hydroxyurea, L-asparaginase, cisplatin, carboplatin, antimony anti-cancer agent, and dacarbazine;
wherein the tumor immunotherapy is one or more of immunomodulator, adoptive cell transfer therapy, tumor-specific vaccine, and small molecule immune drug;
wherein the immunomodulator is one or more of activator of co-stimulatory molecules, immune checkpoint inhibitor, and immune checkpoint activator; wherein the activator of co-stimulatory molecules is one or more of activator of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, and CD83 ligand; wherein the immune checkpoint inhibitor is one or more of inhibitors of PD-1, PD-L1, PD-L2, PD-L3, CTLA4, TIM-3, LAG3, CEACAM-1, CEACAM-3, CEACAM-5, VISTA, VSIR, BTLA, TIGIT, LAIR1, LMTK3, TIGHT, IDO, CD27L, CD40, CD47, CD137, CD160, CD244, CD270, GITR, B7-H1, B7-1, 2B4 and/or TGF-β R;
wherein the adoptive cell transfer therapy is one or more of tumor-infiltrating lymphocyte therapy, engineered T-cell receptor therapy, chimeric antigen receptor T-cell therapy, and NK cell therapy;
wherein the tumor-specific vaccine is one or more of whole tumor cell vaccine, dendritic cell vaccine, gene vaccine, RNA vaccine, and protein peptide vaccine.

19. A pharmaceutical composition, comprising the bifunctional fusion protein of claim 1, or an expression system or a culture of the expression system;
wherein the expression system comprises a construct or a genome incorporating an exogenous polynucleotide, wherein the construct comprises the polynucleotide, and the polynucleotide encodes the bifunctional fusion protein of claim 1.
